(19) United States
(12) Patent Application Publication
(10) Pub. No.: US 2011/0179509 A1
(43) Pub. Date: Jul. 21, 2011

(54) Use of Afamin for Treating Fertility Disorders

(75) Inventors: Hans Dieplinger, Innsbruck (AT); Wolfgang Engel, Gottingen (DE); Georg Wietzorrek, Innsbruck (AT)

(73) Assignee: VITATEQ BIOTECHNOLOGY GMBH, Innsbruck (AT)

(21) Appl. No.: 13/121,881

(22) PCT Filed: Oct. 1, 2009
(86) PCT No.: PCT/AT2009/000377

§ 371 Date: Mar. 30, 2011

(30) Foreign Application Priority Data
Oct. 1, 2008 (AT) A 1535/2008

(51) Int. Cl. A01K 67/027; A61K 38/17; A61K 39/395; A61K 31/7088; A61P 15/00; A61P 15/08

(52) U.S. Cl. 800/18; 514/20.9; 424/172.1; 424/152.1; 424/133.1; 514/44 A; 800/13

(57) ABSTRACT

The invention relates to the use of afamin for the manufacture of a pharmaceutical preparation for the prevention or treatment of fertility disorders.

The invention relates to the use of afamin and afamin-inhibitors.

Vitamin E was discovered in 1922 by Evans and Bishop as a fat-soluble factor necessary for normal reproduction in rat. Since then, the deficiency of vitamin E has been associated with various chronic disorders such as atherosclerosis, ischemic heart disease, immune deficiency, different types of cancer and neurological syndromes that possess a strong oxidative stress component and can be successfully treated with dietary vitamin E supplementation. The central role of vitamin E for maintaining physiological cellular and tissue function has therefore been attributed to two primary functions: as a potent scavenger and antioxidant of reactive oxygen and nitrogen species and, more recently, as a modulator of cellular functions such as adhesion, proliferation and apoptosis.

Due to its hydrophobicity and primary location in the plasma membrane, vitamin E requires special carrier/transport mechanisms in the aqueous environment of plasma, other body fluids and cells. Dietary vitamin E is absorbed, together with lipids, by mucosa cells of the proximal intestine, assembled in the Golgi apparatus into chylomicrons and secreted via lymphatic fluid into the circulation. Vitamin E containing chylomicrons are then partially degraded in plasma to chylomicron remnants and taken up by specific hepatic receptors. In contrast to the unspecific vitamin E uptake by intestinal cells, the liver preferentially incorporates alpha-tocopherol into nascent VLDL, which is secreted into the bloodstream and converted by the lipolytic cascade to LDL and HDL. Vitamin E is delivered preferentially by LDL receptor-mediated uptake to tissues like kidney, adrenal glands, ovary and adipose tissue or, alternatively, via HDL to other tissues with or without lipoprotein internalization.

While the vitamin E transport by the plasma lipoprotein system is well documented and understood (1), little is known about its transport in other body fluids. Human cerebrospinal and follicular fluids lack triglyceride-rich, apolipoprotein B-containing lipoproteins, which are the major vitamin E carriers in plasma. Instead, they contain HDL particles that differ qualitatively and quantitatively from those in human plasma. The transport mechanism of vitamin E in these body fluids is largely unknown. The search for a vitamin E carrier protein revealed the albumin-gene-family member afamin (2, 3, 4, 5).

Afamin is a 75-kDa human serum glycoprotein with vitamin E-binding properties. The afamin gene is located on chromosome 4q11-q13 as part of the albumin gene family. This family consists of human serum albumin (HSA), vitamin D-binding protein (DBP), afamin and alpha-fetoprotein (AFP). Albumin, the most abundant plasma protein, exhibits several functions such as transport and delivery of metabolites and fatty acids as well as regulation of the osmotic pressure in blood. From sequence comparison with DBP it was speculated that afamin might possess sterol-binding properties as well (2, WO95/27059 A1).

Afamin is primarily expressed in the liver and secreted into the plasma. Substantial expression has also been observed in kidney and testes. Significant amounts were detected also in follicular and seminal fluid suggesting possible roles for afamin in vitamin E transport in these body fluids with potential significance for fertility. Since most proteins in human cerebrospinal and follicular fluids originate from plasma, afamin was also purified to homogeneity with vitamin E-binding properties from human plasma using radioactively labelled alpha-tocopherol. Its activity was followed by multi-step chromatography of lipoprotein-depleted plasma (3, 4, 5). Afamin was previously described as a glycoprotein with four or five potential N-glycosylation sites. Glycosylation analysis indicated that >90% of the glycans were sialylated biantennary complex structures.

Afamin plasma concentrations were also found associated with the duration of human pregnancy indicating again a relation between afamin and fertility. Afamin plasma levels of pregnant women rose on average by 50% during the course of a normal pregnancy. Afamin correlates positively with follicle size and maturity.

Afamin is therefore used as a fertility marker (WO01/01148 A1). It is also a tumour marker for tumours of the reproductive organs (WO2006/079136 A1). Due to its specific properties, it can be used for the treatment of oxidative stress, especially in combination with vitamin E (WO02/087604 A2).

Vitamin E has also been demonstrated in follicular, seminal, and cerebrospinal fluid and found associated with ovarian follicle maturation, spermatozoa motility and neurodegenerative disorders like Alzheimer's and Parkinson's disease, emphasizing its central importance for reproductive and neurological functions.

Alpha-fetoprotein, a member of the albumin gene family, was reported to be non-essential for embryonic development but required for female fertility by a knock-out mouse model (6).

In a recently performed study, the vitamin E-binding properties of human afamin were investigated by radioligand assay followed by Scatchard and Hill analysis which revealed binding affinity of afamin for both alpha- and gamma-tocopherol (4). The binding-dissociation constant was determined to be 18 μM, indicating that afamin plays a role as vitamin E carrier in body fluids such as human plasma and follicular fluid under physiological conditions. It was further demonstrated in this study that afamin has multiple binding sites for both alpha- and gamma-tocopherol. Finally, homology modelling and docking calculations on the predicted tertiary structure of afamin were performed demonstrating coincidence between calculated and in vitro results. The vitamin E-binding properties were confirmed using recombinantly expressed afamin.

It is an object of the present invention to provide further uses of afamin and of afamin related metabolism properties.

Therefore, the present invention provides the use of afamin for the manufacture of a pharmaceutical preparation for the prevention or treatment of fertility disorders.

In the course of the present invention further investigations of the role of afamin in fertility were conducted. A relatively high concentration of afamin in follicular fluid lead to the suggestion that afamin has a role in the maturation of follicles. In fact, it could be shown that afamin concentrations in follicular fluid correlated not only with afamin concentrations in plasma, but also with size and therefore maturity of follicles. The vitamin E association of afamin in follicular fluid was directly demonstrated by gel filtration chromatography and immunoprecipitation which confirms the in vitro findings for purified native and recombinant afamin (3, 5).

In order to investigate the physiological role of afamin in detail, gene-knock-out mice were created and characterised. Chimeric (partial afamin-knockout) mice had undetectable afamin blood levels and were completely infertile. Homozygous afamin knock-out animals could therefore not been bred. Histological characterisation of male chimeric animals indicated impaired/dysfunctional spermiogenesis, female animals were histologically free of pathological findings but also infertile.

Supplementation with recombinantly produced murine afamin by a constant diffusion pump device led to restoration of normal testes histology, spermiogenesis and fertility.

To further investigate the role of afamin in fertility, the expression of afamin in normal mice was analysed by RT-PCR in several organ tissues. Aside from the well-known expression of afamin in liver, strong signals were observed in testes and kidney.

These data revealed that afamin is not only a marker for fertility, as demonstrated in WO01/01148 A1, but surprisingly turned out to be a protein with significant influence on fertility properties of an individual. This showed that afamin is also a suitable target to correct deficiencies in fertility or to modulate fertility. A specifically preferred embodiment of the present invention is the prevention of fertility disorders by administration of afamin.

To investigate whether conclusions from these findings in the animal model can be drawn also for human fertility, afamin plasma concentrations were measured in a group of men with various infertility disorders including the Sertoli-Cell-Only Syndrome (SCO). This syndrome is rare but serves as practically relevant and interesting model for the purposes of the present invention, since the testes histology and complete dysfunctional spermiogenesis very much resembles the observed histology of the investigated male afamin knock-out mice. Patients with infertility disorders, including SCO syndrome, had significantly reduced plasma levels of afamin as measured by ELISA.

Accordingly, these findings show that afamin can also be used as a marker for infertility disorders, especially for Non-Obstructive Azoospermia, such as Sertoli-Cell-Only Pattern, Maturation Arrest or Hypospermatogenesis, but also for female fertility disorders, such as Hypothalamic dysfunction, Polycystic Ovarian Syndrome, Anovulation, Poor Ovarian Reserve, Premature Menopause, Luteal Dysgenesis, Endometriosis, Tubal dysfunction, Antisperm Antibodies, Non-Receptive Cervical Mucus and Androgen Insensitivity Syndrome.

These findings, however, also show the relevance of the animal studies of the present invention for the human system, both for the use of afamin to prevent and treat fertility disorders as well as for the use of substances which modulate afamin activity in vivo for the modulation of fertility in humans and animals, especially for contraceptive purposes. In the course of the present invention, afamin knock-out mice were provided which are infertile. However, these mice could successfully be treated with exogenous afamin showing the therapeutic potential for treating human fertility with afamin.

One major aspect of the present invention is therefore the prevention or treatment of fertility disorders. According to the present invention afamin was identified as a key molecule for defining the fertility status of an individual. Influencing the afamin level in vivo directly leads to a change in the fertility status. The animal model according to the present invention clearly showed that afamin is able to treat fertility disorders in an effective manner. It was, however, also shown that afamin can be used to prevent a fertility disorder, so that e.g. genetic disposition (afamin knock-out mutants) is overcome by exogenous afamin supply so that no fertility disorder develops in the animals. According to the results obtained by the animal model according to the present invention and by the measurement of afamin levels in human patients with specific fertility disorders, it follows that the preferred fertility disorders to be prevented or treated with the present invention are those fertility disorders which are associated with an afamin decreased state of the patient. Accordingly, preferred fertility disorders to be prevented or treated are Azoospermiae, especially Sertoli-Cell-Only Pattern, Maturation Arrest or Hypospermatogenesis. Another group of preferred fertility disorders to be prevented or treated according to the present invention consists of Hypothalamic dysfunction, Polycystic Ovarian Syndrome, Anovulation, Poor Ovarian Reserve, Premature Menopause, Luteal Dysgenesis, Endometriosis, Tubal dysfunction, Antisperm Antibodies, Non-Receptive Cervical Mucus and Androgen Insensitivity Syndrome.

Afamin may be applied in any convenient form, for example intravenously, parentally, or locally (in vicinity of testes or ovaries). It may also be applied via implanted (micro-)pumps or via slow release deposits. Usual pharmaceutical formulations and carriers may be used for these purposes and administration routes. For example, for a four week administration of afamin via implanted micro-pumps, 50 micro-g to 1 g afamin per kg body weight of the individual may be applied, preferably 0.1 mg to 100 mg, especially 1 to 10 mg. Daily doses may be preferably designed in the range of 1 micro-g to 50 mg, preferably 10 micro-g to 5 mg, especially 0.1 mg to 1 mg per kg body weight. Preferably, human afamin is delivered to human patients, especially recombinant human afamin. The present invention also includes recombinant variants of albumin with respect to glycosylation patterns, deletion and insertion mutants as equivalents, as long as the afamin activity with respect to fertility is not significantly reduced compared to afamin prepared from human plasma.

Moreover, results from inhibiting afamin in wild-type mice also open the way to a contraceptive application by inhibiting afamin expression in humans.

Therefore, the present invention also relates to the use of an afamin-inhibitor for the manufacture of a contraceptive pharmaceutical preparation. An afamin-inhibitor is a compound which inhibits the in vivo action of afamin in a human or in a (non-human) mammal. Inhibition of afamin expression may be provided by any known compound which inhibits afamin activity, e.g. afamin antibodies (monoclonal as well as polyclonal), afamin siRNA, afamin microRNA, afamin antisense DNA, competitively binding afamin ligands, such as tocopherol derivatives that irreversibly bind with dissociation constants<18 microM (preferably below 10 microM, especially below 1 microM) and inactivate afamin function. The specific sequences for siRNA, antisense, etc. may be derived from public resources, such as the Reference sequences NM 001133 (mRNA) and NP 001124 (protein).

According to preferred embodiments, the afamin-inhibitor is an afamin antibody, especially a humanised monoclonal antibody, or an afamin-antisense nucleic acid. For the purposes of the present invention the term “antibody” also includes functional antibody fragments (such as, Fab, single chain antibodies) and derivatives (especially humanised monoclonal antibodies) as long as the binding specificity and capacity of the “original” antibody is conserved. Administration of the antibodies may be performed with single dosages or long term applications, as outlined above for the administration of afamin. A humanised monoclonal antibody can e.g. be applied in a single dose of 0.01 to 100, preferably 0.1 to 10, especially 0.5 to 5, mg per kg body weight. For long term supplies, for example suppositories comprising a retard formulation of the antibodies with 0.1 mg to 1 g, preferably 1 to 100 mg, per kg body weight may be suitable.

Administration of the compounds according to the present invention to humans or mammals can preferably be achieved by oral or injective administration or by means of diffusion pump devices (similar to “insulin-pumps”).

According to the results obtained in the animal model according to the present invention, specifically inhibition of male fertility is preferred to be achieved in humans. However, since afamin gene deletion resulted in infertility also in female mice, both infertility treatment with exogenous afamin and contraception by inhibiting afamin may work in females as well as in males (humans or mammals).

The chimeric knock-out animals created in the course of the present invention represent highly relevant animal models. The present invention therefore also relates to chimeric afamin knock-out mammals (of course, non-human) which carry no or a non-functioning afamin gene. Preferably, the whole afamin gene is knocked out, or at least 50% of the gene; point or frame shift mutants are possible as well (as long as they lead to a non-functional form of afamin), however, less preferred. The animals according to the present invention are chimeric, not homozygous; however, as shown in the example section, the chimeric knock-outs are already showing the desired lack of fertility property. The animal model according to the present invention is preferably a rodent model, especially a rat or mouse model. A specifically preferred mouse model is described in the example section below and is made by microinjection of ES cells carrying the disrupted afamin allele into mouse embryos (eg. of the C57B1/J mouse strain). Chimeric animals were mated to partners (eg. C57B1/J or 129/Sv partners, respectively) to establish the afamin mutant allele on a hybrid or inbred background (eg. C57B1/J×129/Sv hybrid and on a 129/Sv inbred genetic background).

The present invention is further described in the following examples and the drawing figures, yet without being restricted thereto.

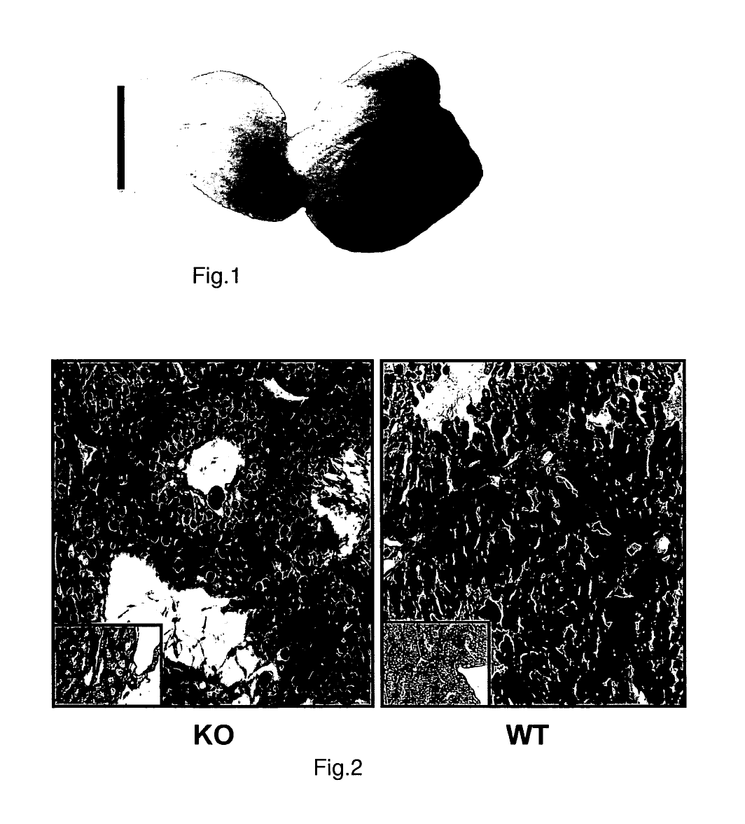

FIG. 1 shows the substantially reduced organ size of testes from a 95% chimeric animal (left) after afamin gene knockout compared to a wildtype mouse (right);

FIG. 2 shows a histological section of testes from the same animals. The chimeric animal (KO) shows most of its testis tissue degenerated with grossly impaired spermatogenesis compared to the wild-type (WT) mouse;

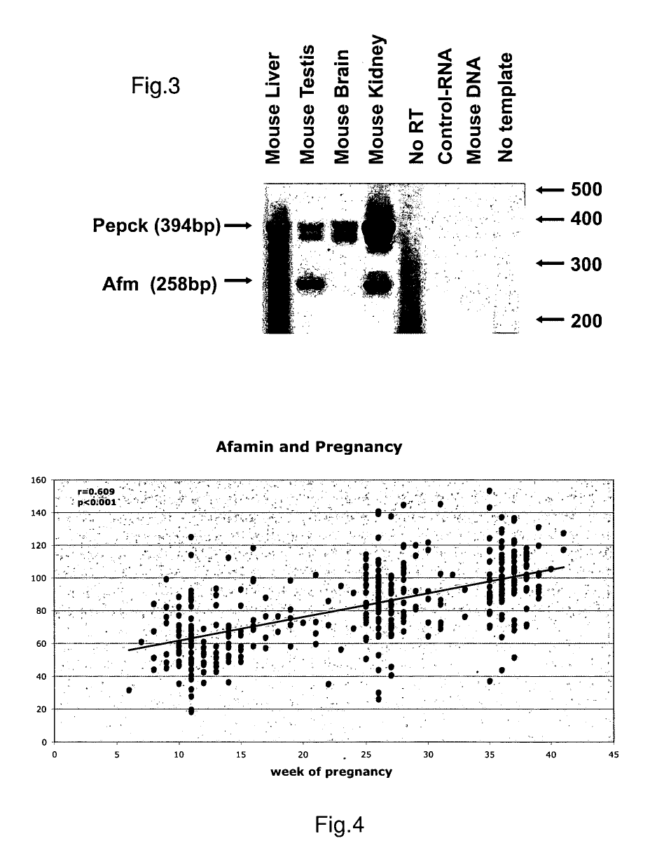

FIG. 3 shows tissue expression of afamin in wild-type mice by RT-PCR; and

FIG. 4 shows afamin plasma concentrations in 400 pregnant women at different time points within their pregnancies.

### EXAMPLES

The following examples result from animal and human studies were performed to characterise in detail a possible causal function of afamin in fertility and reproduction.

### Afamin Knockout Animals.

The first step for the generation of afamin knockout mice was to isolate the genomic fragment containing the mouse afamin gene. For that purpose, the 129/Sv mouse cosmid library from the German resource center (RZPD) was screened with afamin cDNA fragments. After subcloning the restricted fragments into a plasmid vector the restriction map of the afamin locus was determined and confirmed by partial sequencing. For construction of the targeting vector and selection of homologous recombinant embryonal stem (ES) cell clones, the targeted vector was designed by the replacement of internal exons with the Pgk-neo cassette. After transfection of ES cells with the linearised targeted vector, the individual drug-resistance clone was screened for homologous targeting events. An external probe was used to detect the recombinant allele. Rehybridisation with an internal and the neomycin probe confirmed homologous recombination and detected further integration on the targeted vector.

Afamin Chimeric Animals.

Chimeric mice from ES cells carrying the disrupted afamin allele were generated by microinjection of 10-15 ES cells into 3.5-day-old embryos of the C57B1/J mouse strain. Chimeric animals were mated to C57B1/J or 129/Sv partners, respectively, to establish the afamin mutant allele on a C57B1/J×129/Sv hybrid and on a 129/Sv inbred genetic background. Loss of afamin mRNA and protein was checked by Northern and Western blot analysis.

So far 31 chimeric mice (25 males, 6 females) of different degree (20-100%) have been generated. Mice with a high chimeric degree were completely infertile, those with a low degree produced offspring carrying only the wildtype afamin allele. Ovaries and uteruses had normal size and shape whereas the testes of the chimeric mice were significantly smaller compared to wild-type controls (FIG. 1). Histological examinations showed no pathological findings in ovaries and uteri, but highly degenerated testis tissue, mostly affecting sperm producing cells, resulting in a massive impairment (if not dysfunction) of spermatogenesis (FIG. 2).

In order to analyse the afamin concentrations in the partial knock-out mice, an ELISA kit for quantifying afamin in mouse plasma was developed. For that purpose, a polyclonal antibody against mouse afamin was obtained by immunising rabbits with purified recombinant murine afamin. Recombinant expression of murine afamin was achieved by stable transfection of CHO cells with mouse afamin cDNA followed by purification from serum-free culture supernatants. The obtained polyclonal anti-mouse afamin antibody was purified by affinity chromatography on a sepharose column to which recombinant afamin was covalently bound. This affinity-purified antibody was used to coat ELISA-plates and, after conjugation with horse-radish peroxidase, also for detection using an appropriate substrate reaction. Purified recombinant afamin (quantified by amino acid compositional analysis) served as standard. With this ELISA, an afamin plasma concentration in the range of 6-10 mg/l was measured in wild-type mice which is roughly only 10% of respective plasma levels in humans (3). Surprisingly, chimeric afamin knock-out animals of high (close to 100%) chimeric degree had undetectable plasma levels of afamin.

After mating male chimeric animals with wild-type mice, no sperms could be found in uteruses or oviducts, thus resulting in complete infertility. The few fertile low-chimeric mice produced only wild-type offspring indicating no transmission of the recombinant allele.

In order to confirm the role of afamin for fertility, infertile male chimeric animals were substituted with recombinantly produced murine afamin via minipump, which was implanted intraperitonially and released afamin continuously into the organism for 28 days (which corresponds to the time required for spermiogenesis in mice). Control mice received physiological saline solution instead of afamin. At weekly time intervals, plasma was obtained from substituted mice. Plasma concentrations of afamin immediately rose to physiological levels and stayed constant during the infusion period. During the whole afamin substitution period, mice were mated with female wild-type which resulted in several pregnancies and births. Offspring were, however, exclusively wild-type not carrying the recombinant allele. Histological examination of testes after afamin substitution and sacrificing mice showed completely normalised testes tissue and spermiogenesis.

Tissue expression of afamin in normal mice. In order to investigate the observed crucial role of afamin in male infertility in more detail, a detailed expression analysis of afamin was performed in several major organs in normal mice and found, aside from expression in liver and kidney also a strong expression signal in testes in line with the proposed function of afamin in spermiogenesis and fertility (FIG. 3).

Taken together, targeted disruption of the murine afamin gene severely affected spermiogenesis already at the chimeric (“heterozygous”) level. Thus, the vitamin E binding protein afamin plays a central role in (male) fertility.

Male infertility syndromes: Testes of the male chimeric animals seem to resemble the human male infertility phenotype of SCO (Sertoli cell-only) syndrome which is a special form of azospermia. Based on and encouraged by this phenotype of the present animal models a case/control study was initiated by measuring afamin in various male infertility syndromes in comparison with age-matched fertile male controls. These measurements revealed significantly reduced afamin plasma levels (55.7+23.4 mg/l) in infertile patients (n=95) including the rare Sertoli-cell-only (SCO) syndrome (n=9) compared with the fertile control group (71.4+15.3 mg/l, n=95, p<0.001) (Table 1). The SCO syndrome describes a condition of the testes in which only Sertoli cells line the seminiferous tubules. Accordingly, these men show azospermia, which is defined as the absence of sperm in the ejaculate. The prevalence of SCO in the general population is extremely rare. Less than 5-10% of infertile men have SCO. However, diagnosis is possible only by testicular biopsy, and no effective treatment exists.

| | | Afamin plasma | Significance |
|---|---|---|---|
| | | concentration | (total patients |
| Diagnosis | N | (mg/l + SD) | vs controls) |
| Astenozoospermia | 24 | 57.4 + 26.0 | |
| Azoospermia | 22 | 57.2 + 20.2 | |
| Kryptozoospermia | 5 | 69.2 + 28.6 | |
| OAT Syndrome | 35 | 59.0 + 26.3 | |
| SCO | 9 | 50.4 + 15.3 | |
| Total patients | 95 | 55.5 + 23.4 | P < 0.001 |
| Fertile controls | 95 | 71.4 + 15.3 | |

Furthermore, afamin plasma concentrations were measured in 400 pregnant women at different time points within their pregnancies. Afamin correlated positively and significantly with the duration of pregnancy (r=0.609, p<0.001). On average, afamin increased by approximately 100% during a physiological pregnancy (FIG. 4).

In conclusion, the results from animal and human studies according to the present invention show a clear and causal role of afamin in fertility and reproduction. Obtained data not only allow to use afamin as a novel diagnostic marker for human infertility disorders, but also provide a therapeutic potential both to treat human infertility disorders with afamin or to use an afamin-inhibitory principle for a potential male contraceptive medication.

Regarding the infertility treatment, the major known infertility disorders (such as mentioned above) are the primary target for treatment with afamin. These will generally include all those disorders which lead to azospermia (i.e. where no sperm can be detected in the ejaculate).

### REFERENCES

1. Kayden et al., 1993, J. Lipid Res. 34:343-358.
2. Lichenstein et al., 1994, J. Biol. Chem. 269:18149-18154.
3. Jerkovic et al., 2005, J Proteome Res 4:889-899.
4. Voegele et al., 2002, Biochemistry 41:14532-14538.
5. Angelucci et al., 2006, BBA 1764: 1775-1785
6. Gabant et al., 2002, PNAS 99: 12865-12870

1.-8. (canceled)

9. A method of preventing or treating fertility disorders comprising:
obtaining a pharmaceutical preparation comprising obtaining at least one dose of afamin; and
administering at least one dose of afamin to a patient;
wherein a fertility disorder is treated or prevented in the patient.

10. The method of claim 9, wherein the patient has an afamin decreased state.

11. The method of claim 9, wherein the fertility disorder is non-obstructive azoospermia.

12. The method of claim 11, wherein the non-obstructive azoospermia is further defined as sertoli-cell-only pattern azoospermia, maturation arrest, or hypospermatogenesis.

13. The method of claim 9, wherein the fertility disorder is hypothalamic dysfunction, polycystic ovarian syndrome, anovulation, poor ovarian reserve, premature menopause, luteal dysgenesis, endometriosis, tubal dysfunction, antisperm antibodies, non-receptive cervical mucus and/or androgen insensitivity syndrome.

14. The method of claim 9, wherein the dose is a daily dose of 1 μg to 50 mg per kg body weight of the patient.

15. The method of claim 14, wherein the dose is a daily dose of 10 μg to 5 mg per kg body weight of the patient.

16. The method of claim 15, wherein the dose is a daily dose of 0.1 mg to 1 mg per kg body weight of the patient.

17. The method of claim 9, wherein the dose is administered via an implanted micro-pump.

18. The method of claim 9, wherein the afamin is further defined as recombinant human afamin.

19. A method of contraception comprising obtaining an afamin-inhibitor and administering the afamin-inhibitor to a patient.

20. The method of claim 19, wherein the afamin-inhibitor is an afamin antibody.

21. The method of claim 20, wherein the antibody is a monoclonal antibody.

22. The method of claim 21, wherein the monoclonal antibody is humanized.

23. The method of claim 22, wherein the antibody is administered in a dose of 0.01 to 100 mg per kg body weight of the patient.

24. The method of claim 19, wherein the afamin-inhibitor is an afamin-antisense nucleic acid.

25. A non-human chimeric afamin knock-out mammal.

26. The non-human chimeric afamin knock-out mammal of claim 25, further defined as a chimeric afamin knock-out rodent.
